(19) United States
(12) Patent Application Publication
(10) Pub. No.: US 2007/0270807 A1
(43) Pub. Date: Nov. 22, 2007

(54) Multi-piece circumferential retaining ring

(75) Inventors: Rex Armstrong, Cordova, TN (US); Jonathan Dewey, Memphis, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: SDGI Holdings, Inc.

(21) Appl. No.: 11/400,955

(22) Filed: Apr. 10, 2006

(51) Int. Cl. A61F 2/30

(52) U.S. Cl. 606/61

(57) ABSTRACT

Embodiments of retaining rings for use in orthopedic implants, for example, are disclosed. Such embodiments may include two or more pieces that are assembled so that the pieces overlap each other and define an inner diameter that is less than the diameter of a part to be retained. In particular embodiments, the assembled pieces define a complete circle, and they may be substantially planar or include bends, waves or other geometric configurations.

The present disclosure relates to devices and implants used in osteosynthesis and other orthopedic surgical procedures. Specifically, the present disclosure contemplates a multi-piece retaining structure for use with orthopedic implants or other devices.

Several techniques and systems have been developed for correcting and stabilizing damage or malformation of bones, especially the long bones and the spine. In some of these systems, components can be preliminarily linked together, then adjusted with respect to each other and finally locked together. For example, in multi-axial anchor devices, a receiver member and an anchor member (e.g. a screw) may be connected so that the head of the anchor is in the receiver member, and the anchor is multi-axially pivotable with respect to the receiver member. The anchor member must be held or retained against falling out of or through the receiver member, yet must be pivotable until locked. Other types of orthopedic implants have parts that must be held with respect to each other.

Clamps, caps and other devices have been proposed to accomplish such relative holding or retaining. Frequently, however, such devices are relatively bulky and/or provide a relatively high profile on an orthopedic implant or system. Such a high profile can interfere with other parts of an orthopedic implant, or can interfere with or cause damage to tissues adjacent the implant or implant system.

### BRIEF DESCRIPTION OF THE DRAWINGS

FIG. 1 is an exploded perspective view of an embodiment of retaining apparatus.

FIG. 2 is a perspective view of the embodiment illustrated in FIG. 1.

FIG. 3 is a side elevational view of a part of the embodiment illustrated in FIG. 1.

FIG. 4 is a top plan view of the part illustrated in FIG. 3.

FIG. 5 is a perspective view of an embodiment of an orthopedic implant with which the embodiment of FIG. 1 can be used.

FIG. 6 is a partial cut-away view in perspective of the embodiment illustrated in FIG. 5, with an elongated member.

FIG. 7 is a perspective view of an embodiment of part of a retaining apparatus.

FIG. 8 is a perspective view of a retaining apparatus incorporating the embodiment illustrated in FIG. 7.

FIG. 9 is a perspective view of an embodiment of part of a retaining apparatus.

FIG. 10 is a perspective view of an embodiment of part of a retaining apparatus.

FIG. 11 is a perspective view of a retaining apparatus incorporating the embodiment illustrated in FIG. 10.

FIG. 12 is a perspective view of an embodiment of part of a retaining apparatus.

FIG. 13 is a perspective view of a retaining apparatus incorporating the embodiment illustrated in FIG. 12.

FIG. 14 is a perspective view of an embodiment of part of a retaining apparatus.

FIG. 15 is a perspective view of a retaining apparatus incorporating the embodiment illustrated in FIG. 14.

FIG. 16 is a perspective view of an embodiment of part of a retaining apparatus.

FIG. 17 is a perspective view of a retaining apparatus incorporating the embodiment illustrated in FIG. 16.

FIG. 18 is a perspective view of an embodiment of part of a retaining apparatus.

FIG. 19 is a perspective view of a retaining apparatus incorporating the embodiment illustrated in FIG. 18.

FIG. 20 is a perspective view of an embodiment of part of a retaining apparatus.

FIG. 21 is a perspective view of a retaining apparatus incorporating the embodiment illustrated in FIG. 20.

FIG. 22 is a perspective view of an embodiment of part of a retaining apparatus.

FIG. 23 is a perspective view of a retaining apparatus incorporating the embodiment illustrated in FIG. 20.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

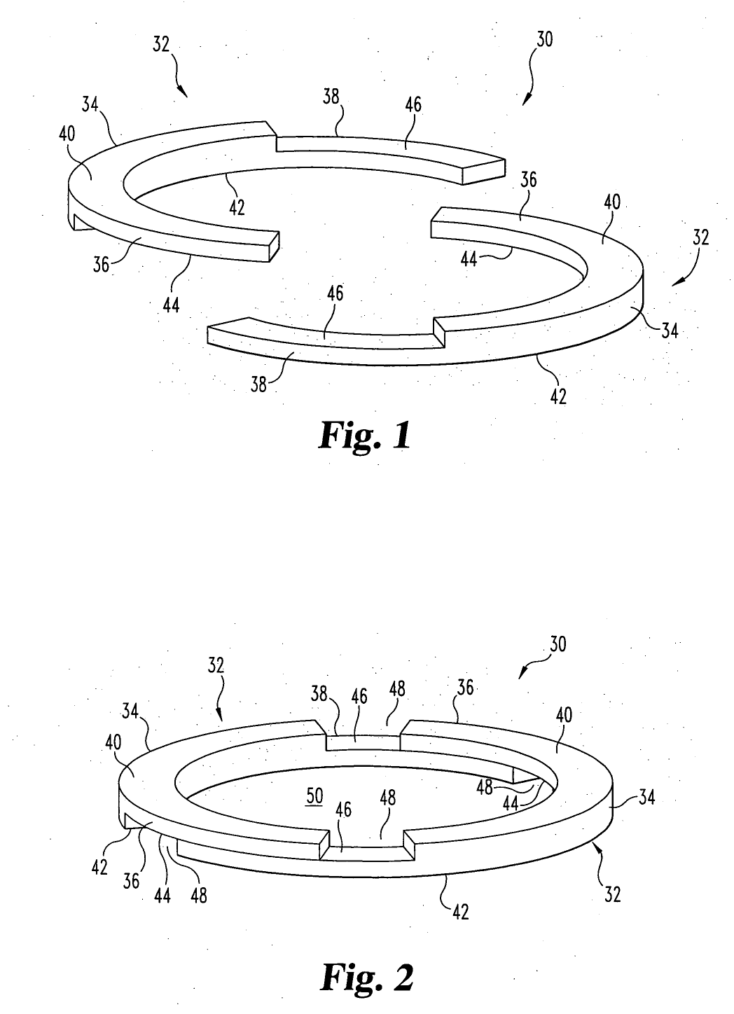

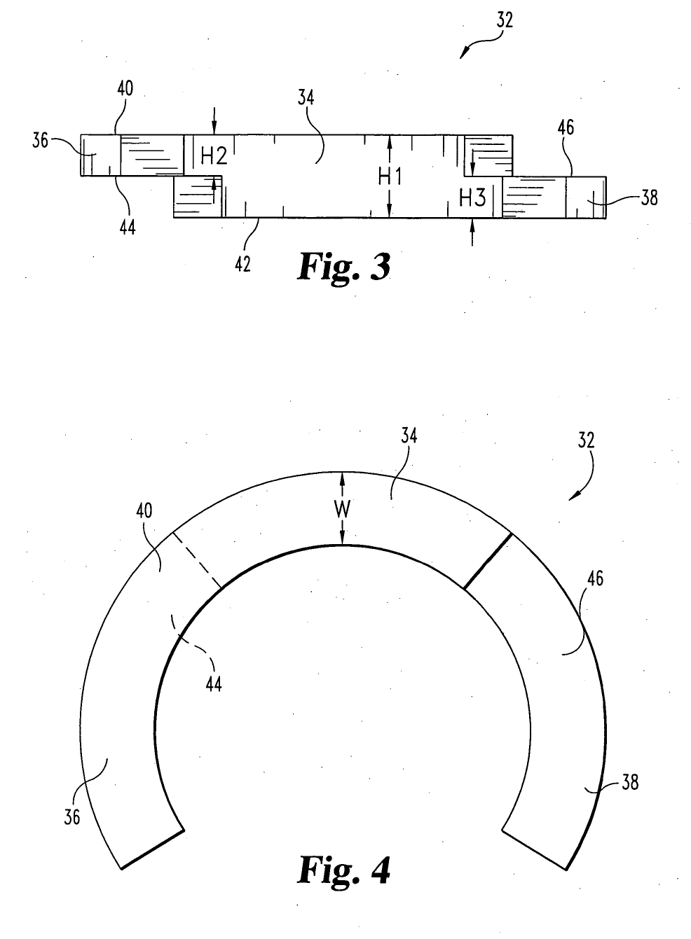

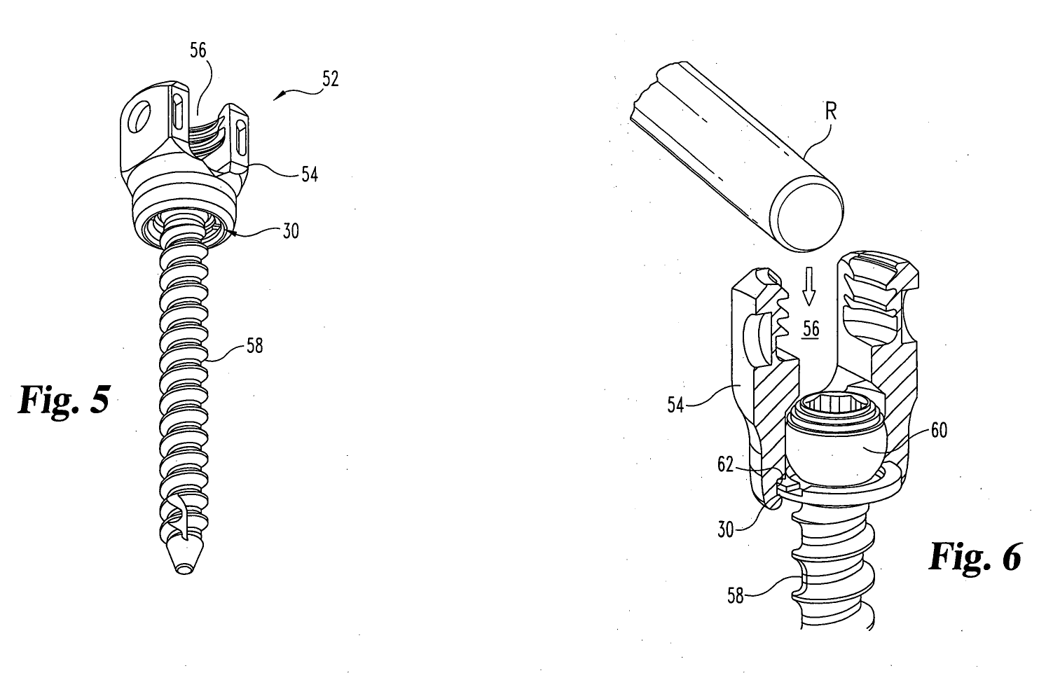

Referring generally to FIGS. 1-6, there is shown an embodiment of a retainer 30. Retainer 30 can be used along with a variety of orthopedic implants or other devices that require connection of one part with another, as further discussed below. In particular cases, retainer 30 can be placed between portions of the two parts so that removal of one of the parts from within the other is impeded or prevented. As shown in FIGS. 5 and 6, an example of such an implant is a multi-axial bone screw having a receiver member and an anchor member, and retainer 30 can be placed in the receiver member and around a portion of the anchor member so that the anchor member is held or retained in the receiver member.

Retainer 30 includes separate pieces 32, which in the illustrated embodiment are substantially identical and generally form part of a circle. A particular embodiment of piece 32 includes a central portion 34, a first arm 36 and a second arm 38, and opposed substantially planar surfaces 40 and 42. Surface 40 extends between arm 36 and central portion 34, and surface 42 extends between arm 38 and central portion 34. Arm 36 and central portion 34 are thus substantially contiguous along surface 40, and arm 38 and central portion 34 are substantially contiguous along surface 42. Each arm 36, 38 is stepped from central portion 34 in this embodiment, and arms 36 and 38 have respective internal surfaces 44 and 46 that, in the illustrated embodiment, are substantially parallel to surfaces 40 and 42. Central portion 34 is the thickest portion, having a height H1 that is substantially the sum of the heights H2 and H3 of arms 36 and 38. In a particular embodiment, heights H2 and H3 are substantially equal, making height H1 substantially twice the value of H2 or H3. Piece 32 has a width W measured along the radius. In the illustrated embodiment, piece 32 extends along an arc of more than 180 degrees from the end of arm 36 to the end of arm 38, and in a particular embodiment that arc is approximately 240 degrees.

As previously noted, the illustrated embodiment of retainer 30 includes two substantially identical pieces 32. As seen in FIGS. 1 and 2, one piece 32 is placed with respect to the other piece 32 so that their respective arms overlap. Thus, surface 44 of each piece 32 abuts at least a portion of surface 46 of the other piece 32, and pieces 32 form a full circle. In the illustrated embodiment, when pieces 32 are abutting to form retainer 30, gaps 48 are present between central portion 34 of each piece 32 and the other piece's arms. Gaps 48 may allow for circumferential adjustment of the relative positions of pieces 32, e.g. pivoting one piece 32 relative to the other piece 32 along the circumference of each, and for collapsing of pieces 32 on themselves for positioning with respect to another device, as further discussed below. Retainer 30, when assembled, provides for an aperture 50 defined by pieces 32.

In use, retainer 30 can be placed in or around parts of an orthopedic implant so as to keep those parts together. As one example, a multi-axial bone implant 52 with which retainer 30 can be used is shown in FIGS. 4 and 5. Implant 52 includes a receiver member 54 having a channel 56 for an orthopedic elongated member (such as a spinal rod) and an anchor 58, such as a screw member with an enlarged head portion 60. Examples of implants with which retainer 30 can be used are shown in U.S. Pat. No. 6,280,442 and U.S. Pat. No. 6,485,491, which are incorporated herein by reference in their entireties. It will be seen that receiver member 54 has a groove 62 proximate to a lower end of receiver member 54, which extends around the entire interior of receiver member 54. Groove 62 has a groove height that is approximately the same as or somewhat larger than height H1 of pieces 32, and a groove width that is slightly less than width W of pieces 32. In this illustrated embodiment, groove 62 is substantially circular in configuration, although it will be seen that groove 62 could have an oblong, oval, square, or other configuration.

In one embodiment, head 60 of anchor 58 is inserted into receiver member 54 so that some or all of head 60 is substantially above groove 62. Retainer 30 is placed in groove 62 and around anchor 58 below head 60. Pieces 32 can be individually inserted into groove 62, or both pieces 32 can be inserted substantially simultaneously, e.g. by placing pieces 32 so that respective surfaces 44 and 46 abut, moving pieces 32 together so that they do not form a circle, moving pieces 32 along anchor 58 and/or to a position adjacent groove 62, and spreading pieces 32 apart so that they enter respective parts of groove 62. Generally speaking, pieces 32 of retainer 30 can be arranged with respect to each other as disclosed herein, e.g. so that a portion of at least one piece 32 overlaps at least a portion of another piece 32, or so that both arms of each piece 32 overlap respective arms of another piece 32, at the same time, prior to or after they are connected to implant 52 so that receiver member 54 and anchor 58 are retained in a relative relationship. Thus, pieces 32 may be seated in groove 62 so that at least a portion of the outer wall of pieces 32 contact receiver member 54. Aperture 50 formed by pieces 32 is smaller than a minimum width or diameter of head 60, in the illustrated embodiment, so that head 60 is impeded or prevented from being pulled through pieces 32 and out of receiver member 54. An elongated member, such as rod R, can be connected with the illustrated embodiment of implant 52, although other types of implants may not be capable of such connection to an elongated member.

Assembly of retainer 30 with an orthopedic implant, such as implant 52, may take place at or after manufacture of the individual parts of the implant and retainer 30, and/or prior to or during a surgical procedure. If such assembly takes place prior to use in surgery, the assembled implant and retainer combination can be moved to a surgical site and placed as desired by the surgeon. Other procedures may be performed and other structure may be connected to the implant. For example, in a case where an implant such as implant 52 is used, the surgeon can insert implant 52 (with retainer 30 retaining anchor 58 in receiver member 54) to a surgical site. Anchor 58 can be attached to bone, e.g. by screwing into a vertebra via a screwdriver or other tool (not shown) inserted through receiver member 54 and into contact with head 60 of anchor 58. When anchor 58 is attached as desired, receiver member 54 is multi-axially pivotable with respect to anchor 58. A rod or other elongated member (not shown) can be inserted into channel 56 and locked to receiver member 54, and receiver member 54 can be locked (with or without intermediate parts) with respect to anchor 58. Other devices, such as connectors, clamps, plates, additional rods, or other implants, can be connected to implant 52 or the rod it is attached to. Distraction, compression or rotation of vertebrae, placement of fusion cages or other devices, or other procedures can be performed before or after placement of implant 52 or placement of a rod with respect to implant 52. When all desired procedures have been completed, the surgeon closes in a manner he or she may prefer.

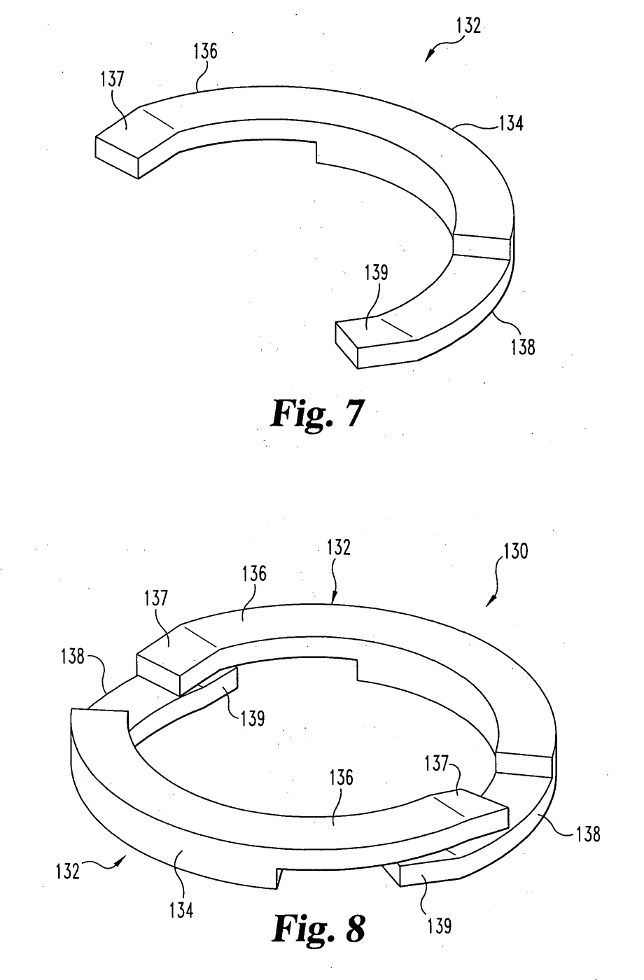

Referring now generally to FIGS. 7-9, a retainer 130 composed of two pieces 132 is quite similar to retainer 30, described above. Pieces 132, are substantially identical and generally form part of a circle in the illustrated embodiment. Central portion 134 and arms 136 and 138 are substantially the same as central portion 34 and arms 36 and 38 described above, with the exception of the end portions 137 and 139 of arms 136 and 138. In the illustrated embodiment, end portions 137 and 139 are bent, with respect to the rest of arms 136 and 138, generally toward central portion 134. The terms “bend” and “bent” used herein refer to a non-coplanar or uneven shape or configuration, but not to a particular method of obtaining such a shape or configuration. When pieces 132 are assembled into retainer 130, each piece's end portion 137 is adjacent the other piece's end portion 139. This nested arrangement of bent end portions 137 and 139 may provide some resistance to circumferential relative movement between pieces 132, where such resistance is desired. The arrangement may also provide for easy relative placement of pieces 132, as portions 137 and 139 may behave similarly to a ball-detent mechanism to indicate proper or preferred positioning of pieces 132 with respect to each other. Further, bent portions 137 and 139 may act as springs, collapsing when retainer 130 is inserted into a groove (e.g. groove 62 described above), and providing further resistance to rotational or other movement of pieces 132 relative to each other or to an implant to which retainer 30 is connected. Thus, bent portions 137 and 139, when nested together, may have a height that is slightly greater than the height of groove 62, so that groove 62 compresses or collapses portions 137 and 139 when retainer 130 is inserted into groove 62.

In other embodiments, only one arm (e.g. arm 136) may have a bent portion (e.g. portion 137), so that bent portion 137 is adjacent an unbent arm 138. Alternatively, arm 136 of one piece 132 may have a bent portion 137, and arm 138 of the other piece 132 may have a bent portion 139, so that one side of retainer 130 has bent portions 137 and 139 facing each other, and another side of retainer 130 has unbent arms 136 and 138 abutting each other. FIG. 9 shows an embodiment in which end portions 137 and 139 have two bends, making a first bent end portion 137a, 137b and a second bend end portion 139a, 139b.

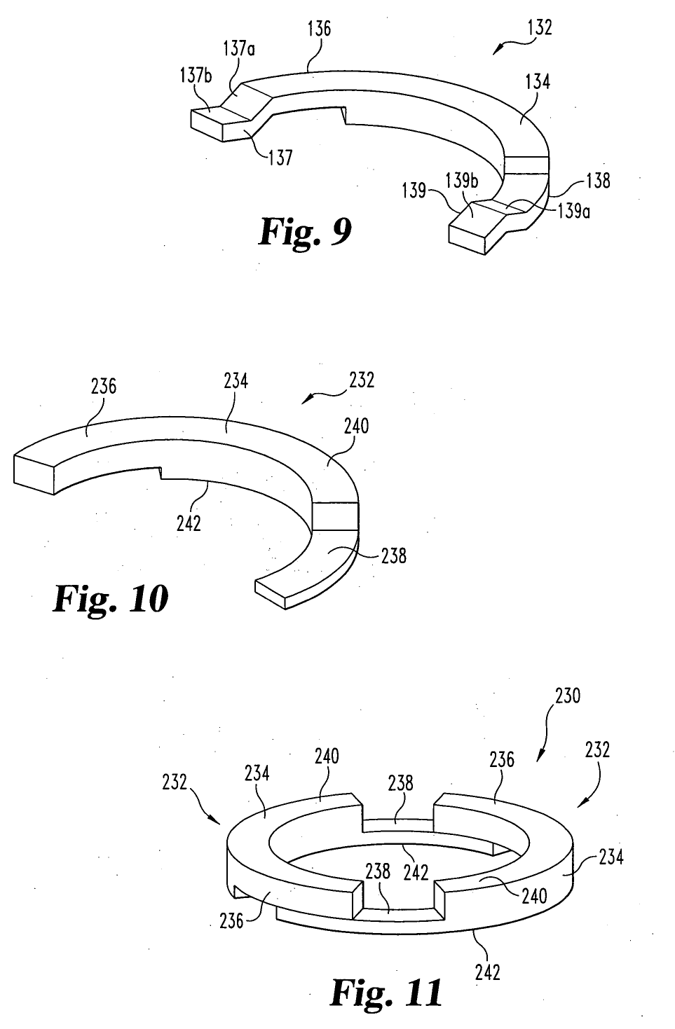

Turning now to FIGS. 10-11, there is shown an embodiment of retainer 230 that is substantially identical in most respects to the embodiment of retainer 30 shown in FIGS. 1-2. Retainer 230 includes separate pieces 232, which in the illustrated embodiment are substantially identical and generally form part of a circle. As with the depicted embodiment of retainer 30, the illustrated embodiment of retainer 230 includes a central portion 234, a first arm 236 and a second arm 238, and opposed substantially planar surfaces 240 and 242. The main difference between retainer 230 and retainer 30 is in the relative thicknesses of arms 236 and 238. Central portion 234 is the thickest portion, having a height that is substantially the sum of the heights of arms 236 and 238. Retainer 230 has arms 236 and 238 with heights that are not equal, with the height of arm 236 being substantially greater than the height of arm 238 in the illustrated embodiment. In one particular embodiment, the height of arm 236 is approximately twice that of arm 238, and thus the height of arm 236 is approximately two-thirds the height of central portion 234, and the height of arm 238 is approximately one-third the height of central portion 234. In other respects, retainer 230 is substantially the same as retainer 30.

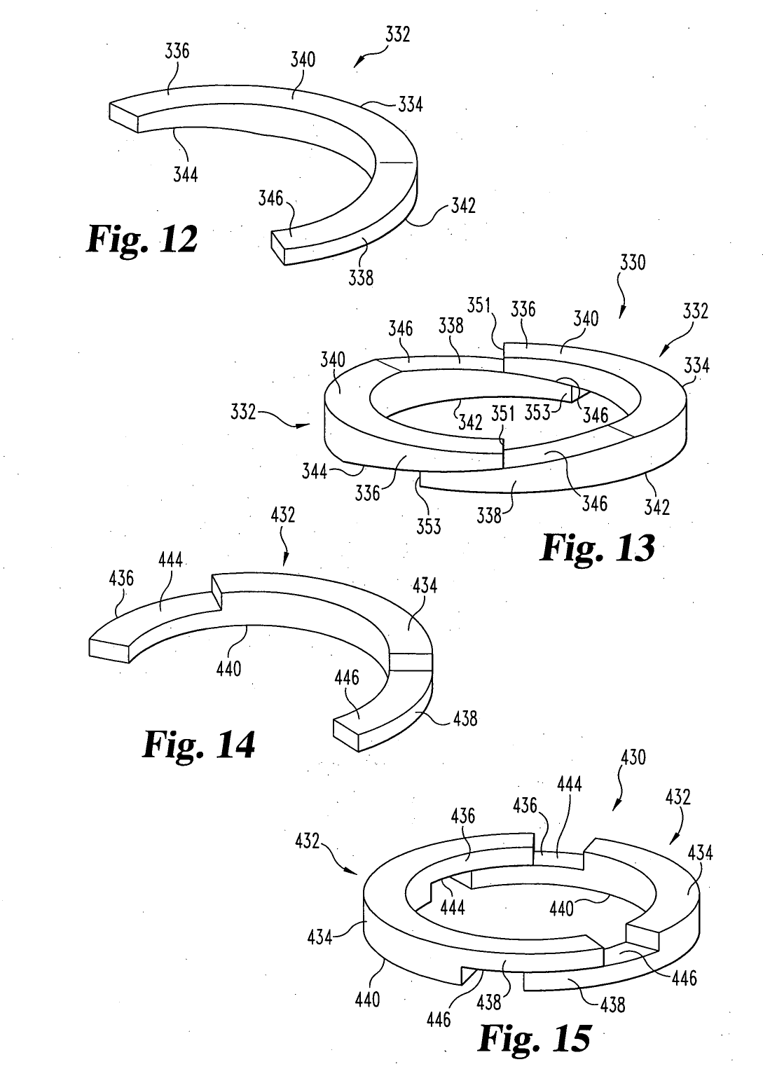

A further embodiment of retainer 330 is illustrated in FIGS. 12-13. Retainer 330 is similar to retainer 30 in many respects and can be used for the same purposes and in the same applications as retainer 30. Retainer 330 includes separate pieces 332, which in the illustrated embodiment are substantially identical and generally form part of a circle. A particular embodiment of piece 332 includes a central portion 334, a first arm 336 and a second arm 338, and opposed substantially planar surfaces 340 and 342. Surface 340 extends between arm 336 and central portion 334, and surface 342 extends between arm 338 and central portion 334. Arm 336 and central portion 334 are thus substantially contiguous along surface 340, and arm 338 and central portion 334 are substantially contiguous along surface 342. Arms 336, 338 have respective surfaces 344 and 346 that are sloped from central portion 334 in this embodiment. Surface 344 meets but is non-planar with surface 342, and surface 346 meets but is non-planar with surface 340. Central portion 334 is the thickest portion, with arms 336 and 338 tapering to relatively thin ends 351 and 353, respectively. In a particular embodiment, the heights of ends 351 and 353 are substantially equal, and are at least slightly less than half of the height of central portion 334. In that embodiment, when pieces 332 are overlapped to make retainer 330 (FIG. 13), the overlapping parts of arms 336 and 338 are approximately the same height as central portion 334. In other respects, retainer 330 is substantially the same as retainer 30.

Referring generally to FIGS. 14-15, there is shown an embodiment of a retainer 430 that is similar in most respects to retainer 30, and is used for the same purposes and in connection with the same types of devices. Retainer 430 includes separate pieces 432, which in the illustrated embodiment are substantially identical and generally form part of a circle. A particular embodiment of piece 432 includes a central portion 434, a first arm 436 and a second arm 438. In this embodiment, a surface 440 that is substantially planar extends from arm 436 through central portion 434 and over to arm 438. Arms 436 and 438 and central portion 434 are thus substantially contiguous along surface 440 in this embodiment. Each arm 436, 438 is stepped from central portion 434 to the same side of central portion 434, so to speak, instead of to opposite sides of a central portion as in the illustrated embodiment of retainer 30. Arms 436 and 438 have respective internal surfaces 444 and 446 that, in the illustrated embodiment, are substantially parallel to surface 440. Arms 436 and 438 may have thicknesses that are approximately equal in one embodiment. Central portion 434 is the thickest portion, having a height that in a particular embodiment is substantially twice the value of the thicknesses of arms 436 and 438. In other respects, retainer 430 is substantially the same as retainer 30.

As previously noted, the illustrated embodiment of retainer 430 includes two substantially identical pieces 432. As seen in FIG. 15, one piece 432 is inverted with respect to the other piece 432, and the two pieces are placed with respect to each other so that their respective arms overlap. Thus, the surfaces 444 of each piece 432 abut at least a portion of each other, the surfaces 446 of each piece 432 abut at least a portion of each other, and pieces 432 form a full circle.

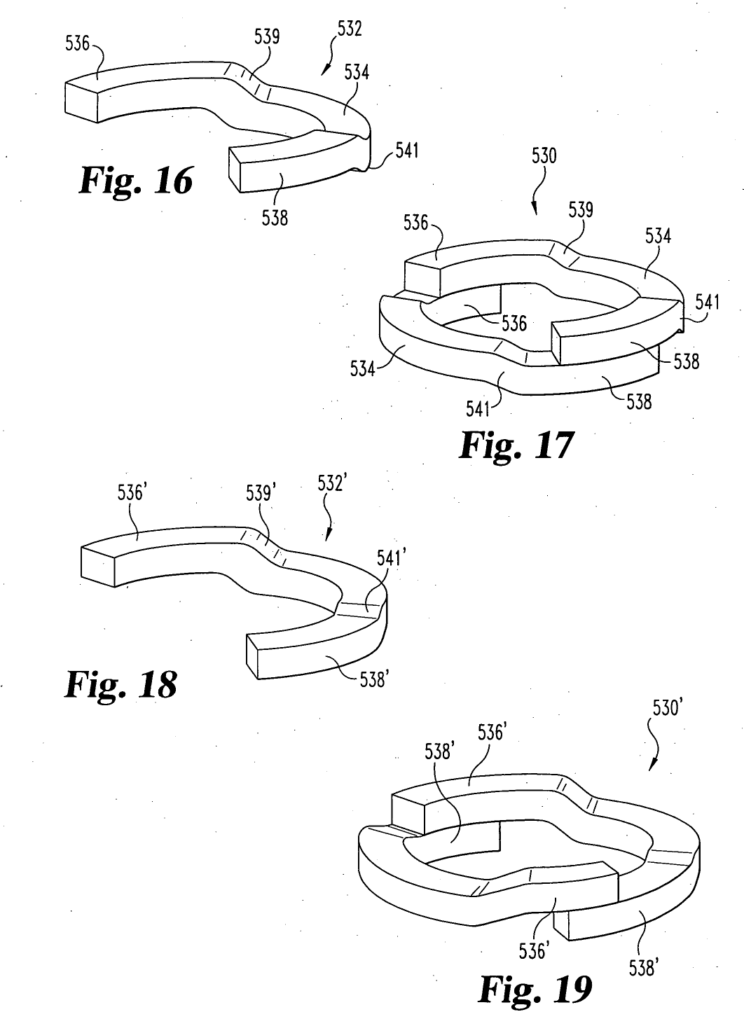

Referring now generally to FIGS. 16 and 17 there is shown a further embodiment of a retainer 530 having similarities to the illustrated embodiment of retainer 430, and which can be used for the same purposes and with similar devices to those with which other retainer embodiments disclosed herein are used.

Retainer 530 includes separate pieces 532, which in the illustrated embodiment are substantially identical and generally form part of a circle. A particular embodiment of piece 532 includes a central portion 534, a first arm 536 and a second arm 538. Each of arms 536 and 538 and central portion 534 are substantially identically shaped and sized in this illustrated embodiment. A first S-like bend 539 is between arm 536 and central portion 534, and a second S-like bend 541 is between arm 538 and central portion 534. Arms 536 and 538 and central portion 534 are thus wavy, having upper and lower surfaces that are contiguous from arm 536, through S-bend 539, central portion 534 and S-bend 541, to arm 538. In other respects, retainer 530 is substantially the same as retainer 430.

As previously noted, the illustrated embodiment of retainer 530 includes two substantially identical pieces 532. As seen in FIG. 17, one piece 532 is inverted with respect to the other piece 532, and the two pieces are placed with respect to each other so that their respective arms overlap. Thus, arms 536 and 538 of a first piece 532 each overlap and abut the respective arms 536 and 538 of the other piece 532, and pieces 532 form a full circle. Arms 536 and 538 are shown generally to the same side of central portion 534 in this illustrated embodiment. That is, referring to the top of piece 532 as seen in FIG. 16 for convenience, there is an up-slope at 539 and 541 between central portion 534 and each of arms 536 and 538. In other embodiments (e.g. retainer 530′ in FIGS. 18-19) arms such as arms 536′ and 538′ may be on generally different sides of a central portion such as central portion 534′. That is, referring to the top of piece 532′ as seen in FIG. 18 for convenience, there is an up-slope at 539 between central portion 534′ and arm 536′, and a down-slope 541′ between central portion 534′ and arm 538. Two such pieces 532′ can be placed together to form retainer 530′, which in other respects is similar or identical to retainer 530 or other retainer embodiment disclosed herein.

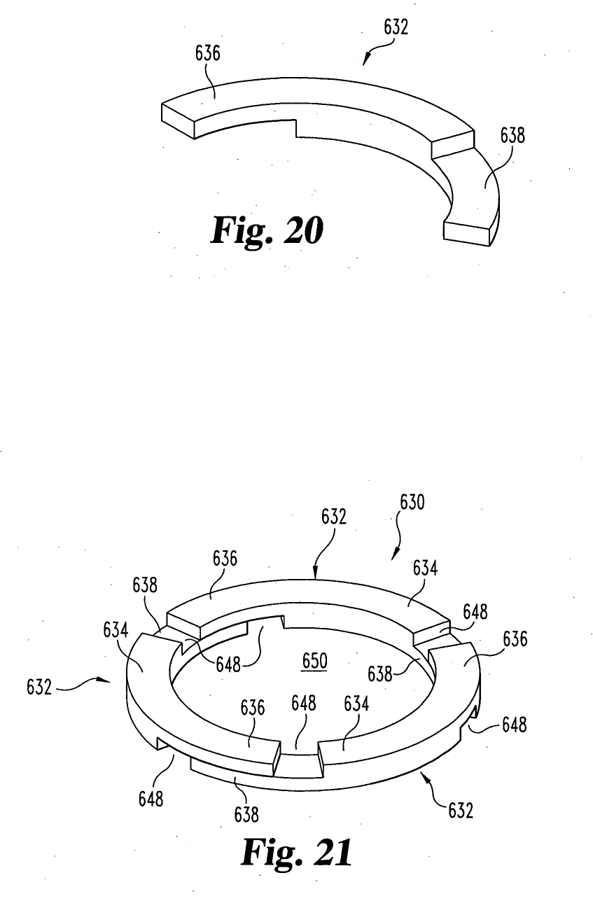

FIGS. 20 and 21 show a further embodiment of a retainer 630 that is essentially identical to the illustrated embodiment of retainer 30, except that retainer 630 uses three substantially identical pieces 632. Pieces 632 are substantially the same as pieces 32, described above, having arms 636 and 638 that are substantially the same as arms 36 and 38, and for brevity's sake that description is not repeated here. However, pieces 632 cover somewhat less of an arc than pieces 32 cover. In the illustrated embodiment, pieces 632 extend along an arc of more than 120 degrees from the end of one arm 636 to the end of arm 638, and in a particular embodiment that arc is approximately 135 degrees or more.

As previously noted, the illustrated embodiment of retainer 630 includes substantially identical pieces 632. As with other embodiments, one piece 632 is placed with respect to the other piece 632 so that their respective arms overlap. Thus, arm 636 of each piece 632 abuts at least a portion of arm 638 of a neighboring piece 632, and pieces 632 form at least substantially a full circle. In the illustrated embodiment, when pieces 632 are abutting to form retainer 630, gaps 648 are present between central portion 634 of each piece 632 and a neighboring piece's arms. Gaps 648 may allow for circumferential adjustment of the relative positions of pieces 632, e.g. pivoting one piece 632 relative to the other piece 632 along the circumference of each, or for collapsing of pieces 632 for assembly with other device(s), substantially as described above. Retainer 630, when assembled, provides for an aperture 650 defined by pieces 632.

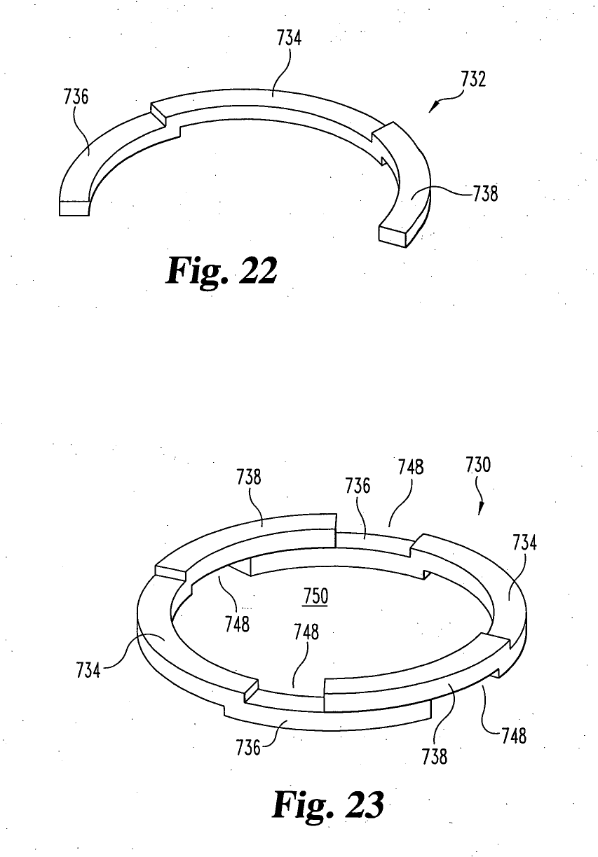

FIGS. 22 and 23 show a further embodiment of a retainer 730 that is quite similar to other illustrated embodiments of a retainer. Retainer 730 includes two substantially identical pieces 732. Pieces 732 each have a central portion 734 connected to arms 736 and 738, much as described above with respect to piece 32. In the illustrated embodiment of piece 732, central portion 734 is substantially of the same thickness as each arm 736 and 738, and is stepped from each arm 736 and 738. Thus, as seen in FIG. 22, as one moves from arm 736 to central portion 734, there is a step up, and as one moves from central portion to arm 738, there is another step up. Piece 732 may be thought of as the same as piece 32, but having a central portion 734 with cut-outs on top and bottom, eliminating the common surfaces central portion 34 shares with arms 36 and 38 in the illustrated embodiment of piece 32.

As with other embodiments, one piece 732 is placed with respect to the other piece 732 so that at least one of their respective arms overlap to form retainer 730. Thus, in the illustrated embodiment arm 736 of each piece 732 abuts at least a portion of arm 738 of the other piece 732, so that pieces 732 form at least substantially a full circle. When pieces 732 are abutting or adjacent to form retainer 730, gaps 748 are present between central portion 734 of each piece 732 and a neighboring piece's arms. Gaps 748 may allow for circumferential adjustment of the relative positions of pieces 732, e.g. pivoting one piece 732 relative to the other piece 732 along the circumference of each, or for collapsing of pieces 732 for assembly with other device(s), substantially as described above. Retainer 730, when assembled, provides for an aperture 750 defined by pieces 732.

Retainers as disclosed herein may be made out of stainless steel, titanium, certain hard plastics or ceramics, among other materials. It will be recognized that any sturdy biocompatible material may be used to construct the disclosed retainers.

Exemplary embodiments of retainers have been disclosed above. It will be seen that modifications may be made to those embodiments. For example, the inside edges of the illustrated embodiments of retainers (e.g. the edges adjacent opening 50 in retainer 30) are generally depicted as sharp corners. In other embodiments, such inside edges of the constituent pieces of the retainers may have curved (e.g. spherical), conical, sloped or other surfaces to accommodate pivoting of a bone screw or other part. Similarly, surface 40 of retainer 30, or analogous surfaces of other embodiments of retainers disclosed herein, could be curved or rounded, such that a cross section through an arm or central portion of such a retainer would show an arced, part-circular or wholly circular shape.

Retainers as disclosed herein may be used with a number of different apparatus for which it is desired to retain or hold one part within or with respect to another. Such apparatus may include various embodiments of bone screws, as discussed above, as well as orthopedic hooks, clamps, bolts, connectors, prostheses, fusion devices, and the like.

It is intended that various parts of the embodiments disclosed above can be used with or incorporated into other embodiments. As one example, the “bent” ends shown in FIGS. 7-9 and described in the text may be used with other embodiments, such as the “same side” embodiment of FIGS. 14-15. Further, the description above notes that in several embodiments identical pieces are used to make a particular retainer. It is intended that unlike pieces may be used to create a retainer. As one particular example, a piece 32 could be used with a piece 132, a piece 232, or other pieces to create what could be called a hybrid retainer.

It will also be seen that the illustrated retainer embodiments generally overlap on two sides so that the retainer forms a complete circle. It is possible to have two pieces (e.g. pieces 32) sized and configured so that they need only overlap on one side, or potentially not overlap at all, and still operate together to retain one part within or with respect to another. For example, in a construct such at that shown in FIGS. 5 and 6, the dimensions of pieces 32 can be chosen with respect to the given dimensions of receiver member 54, head portion 60 and groove 62 so that pieces 32 overlap on one side or not at all when placed in groove 62, yet the diameter of the opening between pieces 32 is smaller than the diameter of head portion 60. In such a situation, even though pieces 32 do not form an entire circle, or in cases in which pieces 32 form an oblong or non-circular interior opening, they still perform as a retainer to hold head portion 60 in receiver member 54. Similarly, it is not necessary to have the outer profile of retainer as disclosed herein be strictly circular. As long as an opening between its respective pieces is smaller than a portion of an anchor, a retainer as disclosed above may be oblong, oval, partially circular or otherwise configured. Thus, where a groove (such as groove 62) is non-circular, a retainer embodiment in that groove need not necessarily have a circular outer profile.

While the disclosure has been illustrated and described in detail in the drawings and foregoing description, the same is to be considered as illustrative and not restrictive in character, it being understood that only illustrated embodiments have been shown and described and that all changes and modifications that come within the spirit of the disclosure are desired to be protected.

What is claimed is:

1. A retaining ring for use with orthopedic medical apparatus, comprising:
a first arcuate piece having a central portion, a first arm, and a second arm; and
a second arcuate piece having a central portion, a first arm, and a second arm, said second arcuate piece being non-integral with said first arcuate piece;
wherein said first and second pieces are arranged so that at least part of one of said arms of said first element overlaps at least one of said arms of said second element.

2. The apparatus of claim 1, wherein said first arm of said first piece overlaps said second arm portion of said second piece, and said second arm of said first piece overlaps said first arm of said second piece.

3. The apparatus of claim 1, further comprising a third arcuate piece, said third piece having a central portion, a first arm and a second arm.

4. The apparatus of claim 3, wherein said first arm of said first piece overlaps at least part of said first arm of said second piece, said second arm of said second piece overlaps at least part of said first arm of said third piece, and said second arm of said third piece overlaps at least part of said second arm of said first piece.

5. The apparatus of claim 1, wherein said first and second pieces are substantially identical.

6. The apparatus of claim 1, wherein said arms of at least one of said pieces have a height that is substantially equal, and said central portion of said at least one of said pieces has a height that is approximately twice said height of said arms.

7. The apparatus of claim 1, wherein said central portion of at least one of said pieces has a first surface contiguous with said first arm of said at least one of said pieces, and a second surface opposed to said first surface and contiguous with said second arm of said at least one of said pieces.

8. The apparatus of claim 1, wherein said central portion of at least one of said pieces has a surface contiguous with said first arm and said second arm of said at least one of said pieces.

9. The apparatus of claim 1, wherein said central portion of at least one of said pieces includes a surface, and at least one of said arms of said at least one of said pieces includes a portion that is non-coplanar with respect to said surface.

10. The apparatus of claim 9, wherein said at least one of said arms has an end portion with at least one bend.

11. The apparatus of claim 9, wherein said at least one of said arms has an end portion with at least two bends.

12. The apparatus of claim 9, wherein substantially all of said at least one of said arms is sloped with respect to said surface.

13. The apparatus of claim 1, wherein at least one of said arms of said first piece nests inside at least one of said arms of said second piece, said nesting providing at least one of resistance to relative rotational movement between said pieces and an indication of proper relative positioning of said pieces.

14. The apparatus of claim 1, further comprising an anchor member having a head, said head having a diameter, wherein a distance between said first and second pieces is smaller than said diameter of said head.

15. The apparatus of claim 14, further comprising an elongated member connected to said receiver member.

16. An orthopedic medical apparatus, comprising:
a receiver member having a lower opening and a groove around said lower opening;
a first retaining piece having a central portion, a first arm and a second arm; and
a second retaining piece having a central portion, a first arm and a second arm, said second retaining piece being non-integral with said first retaining piece;
wherein said retaining pieces are each at least partially within said groove.

17. The apparatus of claim 16, wherein at least one of said arms of said first retaining piece and at least one of said arms of said second retaining piece at least partially overlap.

18. The apparatus of claim 16, wherein said first arm of said first retaining piece overlaps at least part of said first arm of said second retaining piece, and said second arm of said first retaining piece overlaps at least part of said second arm of said second retaining piece.

19. The apparatus of claim 16, further comprising a third retaining piece, said third retaining piece having a central portion, a first arm and a second arm.

20. The apparatus of claim 19, wherein said first arm of said first retaining piece overlaps at least part of said first arm of said second retaining piece, said second arm of said second retaining piece overlaps at least part of said first arm of said third retaining piece, and said second arm of said third retaining piece overlaps at least part of said second arm of said first retaining piece.

21. The apparatus of claim 16, wherein said first and second retaining pieces are substantially identical.

22. The apparatus of claim 16, wherein said arms of at least one of said retaining pieces have a height that is substantially equal, and said central portion of said at least one of said retaining pieces has a height that is approximately twice said height of said arms.

23. The apparatus of claim 22, wherein said groove has a height, and said height of said central portion is approximately equal to or less than said height of said groove.

24. The apparatus of claim 16, wherein said central portion of at least one of said retaining pieces has a first surface contiguous with said first arm of said at least one of said retaining pieces, and a second surface opposed to said first surface and contiguous with said second arm of said at least one of said retaining pieces.

25. The apparatus of claim 16, wherein said central portion of at least one of said retaining pieces has a surface contiguous with said first arm and said second arm of said at least one of said retaining pieces.

26. The apparatus of claim 16, wherein said central portion of at least one of said retaining pieces includes a surface, and at least one of said arms of said at least one of said retaining pieces includes a portion that is sloped with respect to said surface.

27. The apparatus of claim 26, wherein said at least one of said arms has an end portion with at least one bend.

28. The apparatus of claim 26, wherein said at least one of said arms has an end portion with at least two bends.

29. The apparatus of claim 26, wherein substantially all of said at least one of said arms is sloped with respect to said surface.

30. The apparatus of claim 16, further comprising an anchor member having a head, said head having a diameter, wherein a distance between said first and second retaining pieces is smaller than said diameter of said head.

31. The apparatus of claim 30, further comprising an elongated member connected to said receiver member.

32. A method comprising:
providing a first arcuate retaining piece and a second arcuate retaining piece;
arranging said first and second pieces so that a portion of at least one of said pieces overlaps at least a portion of the other of said pieces;
connecting said first and second pieces to an orthopedic medical device having a first part and a second part, so that said first part and said second part are retained in a relative relationship.

33. The method of claim 32, wherein said arranging and said connecting step are performed at substantially the same time.

34. The method of claim 32, wherein said arranging step results in said first and second pieces overlapping at two portions.

35. The method of claim 32, wherein said first part of said orthopedic medical device is a receiver member and said second part of said orthopedic medical device is a bone anchor having a head, and said connecting step includes connecting said pieces to said receiver member and around said bone anchor so that said bone anchor head cannot pass said pieces.

36. The method of claim 32, wherein said orthopedic medical device is an implant, and further comprising connecting an elongated member to said implant.
